(19) United States
(12) Patent Application Publication
(10) Pub. No.: US 2026/0134766 A1
(43) Pub. Date: May 14, 2026

(54) FIRE DETECTION APPARATUS AND METHOD USING MULTIMODAL GAS SENSOR ARRAY

(71) Applicant: Electronics and Telecommunications Research Institute, Daejeon (KR)

(72) Inventors: Dae Sik LEE, Daejeon (KR); Jun Yeong LEE, Daejeon (KR)

(21) Appl. No.: 19/344,102

(22) Filed: Sep. 29, 2025

(30) Foreign Application Priority Data
Sep. 27, 2024 (KR) 10-2024-0131896

(51) Int. Cl. G08B 17/117; A62C 37/36; A62C 99/00; G01N 33/00

(52) U.S. Cl. CPC G08B 17/117; A62C 37/36; G01N 33/0006; G01N 33/0022; G01N 33/004; G01N 33/0047; G01N 33/005; G01N 33/0068; A62C 99/0081

(57) ABSTRACT

The present invention relates to a fire detection apparatus using a multimodal gas sensor array, and the fire detection apparatus includes a multimodal gas sensor array configured to detect gas information when a fire occurs, and a processor configured to analyze a gas detection pattern detected through the multimodal gas sensor array to determine occurrence of the fire and a type of the fire.

### CROSS-REFERENCE TO RELATED APPLICATION

This application claims priority to and the benefit of Korean Patent Application No. 10-2024-0131896, filed on Sep. 27, 2024, the disclosure of which is incorporated herein by reference in its entirety.

### BACKGROUND

### 1. Field of the Invention

The present invention relates to a fire detection apparatus and method using a multimodal gas sensor array, which are capable of quickly and accurately detecting a fire using the multimodal gas sensor array.

### 2. Discussion of Related Art

Typically, a fire detection apparatus may be attached to a wall or a ceiling.

A fire detection apparatus may determine whether a fire has occurred at a position at which the fire detection apparatus is installed by using whether a temperature has risen to a threshold or higher, whether smoke has been detected, and whether combustion gases (for example, carbon monoxide) have been detected at a certain concentration or more.

In an initial stage of fire occurrence, very small amounts of specific gases (or odors) are generated.

Conventionally, fire occurrence has been monitored using a single gas sensor having sensitivity to minute gas leakage or gases generated in an initial stage of fire occurrence and having selectivity for detectable gases (that is, a sensor that detects only specific gas leakage).

However, when fire occurrence is detected using a single gas sensor, there is a problem that a fire cannot be quickly and accurately detected according to a position at which the fire has occurred (for example, a house, a factory, an energy storage system (ESS), or an electric vehicle), an object in which the fire has occurred (for example, wood, a synthetic compound, a chemical substance, paper, or a battery), and a fire range (for example, a small fire range in an initial stage of the fire).

In this way, when a fire cannot be quickly and accurately detected in an initial stage of fire occurrence, there is a problem that human and material damage further increases as the fire spreads.

Therefore, there is a need for detection technology that can quickly and accurately detect a fire even in an initial stage of fire occurrence.

### SUMMARY OF THE INVENTION

The present invention is directed to providing a fire detection apparatus and method using a multimodal gas sensor array, which are capable of quickly and accurately detecting a fire using the multimodal gas sensor array.

According to an aspect of the present invention, there is provided a fire detection apparatus using a multimodal gas sensor array, including a multimodal gas sensor array configured to detect gas information when a fire occurs, and a processor configured to analyze a gas detection pattern detected through the multimodal gas sensor array to determine occurrence of the fire and a type of the fire.

The multimodal gas sensor array may be implemented as sensor arrays having different sensitivities or gas sensor arrays operating in different modes or in different manners.

The processor may detect the fire based on the gas detection pattern that is detected by each gas sensor of a gas sensor array implemented in the multimodal gas sensor array.

The gas sensor array may comprise a hydrogen sensor, a carbon monoxide sensor, a carbon dioxide sensor, and a VOCs (Volatile Organic Compounds) sensor.

The hydrogen sensor may detect gas at a level of 1-100 ppm, the carbon monoxide sensor may detect gas at a level of 1-100 ppm, the carbon dioxide sensor may detect gas at a level of 1-1000 ppm, and the VOCs (Volatile Organic Compounds) sensor may detect gas at a level of 1-500 ppm.

The processor may determine the type of the fire using information detected by the multimodal gas sensor array, and the type of the fire may include at least one of a position at which the fire has occurred, an object in which the fire has occurred, and a fire range.

By using an artificial intelligence mode, the processor may pre-learn a gas detection pattern of gas, which various types of gases having various intensities are mixed, generated when the fire occurs, and even when there is a difference in sensing sensitivity between respective gas sensors included in the multimodal gas sensor array, gas detection patterns detected by the respective gas sensors may have a similar form within an error range.

The fire detection apparatus may further include a fire prevention signal output module configured to output a fire prevention signal to at least one fire prevention device for fire response, which is designated in advance, when the processor determines that the fire has occurred.

The fire protection device may include an alarm device, an air purification device, and a fire extinguishing device configured to spray a fire extinguishing liquid or fire extinguishing powder corresponding to an object in which the fire has occurred, or the type of the fire.

The processor may analyze the gas detection signal pattern detected through the multimodal gas sensor array based on pre-performed learning to determine the occurrence of the fire and the type of the fire, may operate an alarm device and an air purification device through a fire protection signal output module when the occurrence of the fire and the type of the fire are determined, and may also perform fire protection by automatically providing a notification corresponding to the type of the fire to a fire extinguishing device and automatically driving the fire extinguishing device.

The processor may pre-learn a gas detection pattern that is generated according to the type of the fire provided by a learning device.

The learning device may generate individual data for each type of gas, may generate gas combination data for each type of fire occurrence, may generate gas measurement data for each type of fire occurrence, and may synthesize gas combination data generated according to the type of the fire and gas measurement data over time to generate a gas detection pattern that is generated according to the type of the fire.

According to another aspect of the present invention, there is provided a fire detection method using a multimodal gas sensor array, including detecting, by a processor, gas information through a multimodal gas sensor array when a fire occurs, and analyzing, by the processor, a gas detection pattern detected through the multimodal gas sensor array and determining occurrence of the fire and a type of the fire.

In the detecting of the gas information when the fire occurs, the multimodal gas sensor array may be implemented as sensor arrays having different sensitivities or gas sensor arrays operating in different modes or in different manners.

In the determining of the occurrence of the fire and the type of the fire, the processor may detect the fire based on a gas detection pattern that is detected by each gas sensor of a gas sensor array implemented in the multimodal gas sensor array.

In the determining of the occurrence of the fire and the type of the fire, the processor may determine the type of the fire using information detected by the multimodal gas sensor array, and the type of the fire may include at least one of a position at which the fire has occurred, an object in which the fire has occurred, and a fire range.

In the determining of the occurrence of the fire and the type of the fire, by using an artificial intelligence mode, the processor may pre-learn a gas detection pattern of gas, which various types of gases having various intensities are mixed, generated when the fire occurs, and even when there is a difference in sensing sensitivity between respective gas sensors included in the multimodal gas sensor array, gas detection patterns detected by the respective gas sensors may have a similar form within an error range.

The fire detection method may further include, after the determination of whether the fire has occurred, and the type of the fire, outputting, by a fire prevention signal output module, a fire prevention signal to at least one fire prevention device for fire response, which is designated in advance, when the processor determines that the fire has occurred.

In the outputting of the fire prevention signal to the at least one fire prevention device, the fire protection device may include an alarm device, an air purification device, and a fire extinguishing device configured to spray a fire extinguishing liquid or fire extinguishing powder corresponding to an object in which the fire has occurred, or the type of the fire.

In the determining of the occurrence of the fire and the type of the fire, the processor may analyze a gas detection signal pattern detected through the multimodal gas sensor array based on pre-performed learning to determine the occurrence of the fire and the type of the fire, may operate an alarm device and an air purification device through a fire protection signal output module when the occurrence of the fire and the type of the fire are determined, and may also perform fire protection by automatically providing a notification corresponding to the type of the fire to a fire extinguishing device and automatically driving the fire extinguishing device.

Before the determining of the occurrence of the fire and the type of the fire, the processor may pre-learn a gas detection pattern that is generated according to the type of the fire provided by a learning device.

In order for the processor to pre-learn the gas detection pattern, the learning device may generate individual data for each type of gas, may generate gas combination data for each type of fire occurrence, may generate gas measurement data for each type of fire occurrence, and may synthesize gas combination data generated according to the type of the fire and gas measurement data over time to generate a gas detection pattern that is generated according to the type of the fire.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is an exemplary view illustrating a schematic configuration of a fire detection apparatus using a multimodal gas sensor array according to one embodiment of the present invention;

FIG. 2 is an exemplary view for describing the features of a gas detection pattern of a multimodal gas sensor array in FIG. 1;

FIG. 3 is an exemplary view for describing an operating principle of the multimodal gas sensor array in FIG. 1;

FIG. 4 is an exemplary view for describing a driving method in which artificial intelligence is introduced into the multimodal gas sensor array in FIG. 1;

FIG. 5 is an exemplary view for describing an additional module for expanding a function of the multimodal gas sensor array in FIG. 1;

FIG. 6 is a flowchart for describing a fire detection method using a multimodal gas sensor array according to one embodiment of the present invention; and

FIG. 7 is a flowchart for exemplarily describing a method of training a processor with a method of detecting a type of fire in FIG. 6.

### DETAILED DESCRIPTION OF EXEMPLARY EMBODIMENTS

Hereinafter, embodiments of a fire detection apparatus and method using a multimodal gas sensor array according to embodiments of the present invention will be described.

The accompanying drawings are not necessarily to scale and in some instances, proportions may have been exaggerated in order to clearly illustrate features of the embodiments. Further, the terms to be described below are terms defined in consideration of functions in the present invention and thus may vary according to intentions or customs of users and operators. Accordingly, the definitions of such terms should be made based on the content throughout the specification.

Hereinafter, embodiments of the present invention will be described in detail with reference to the accompanying drawings so that those skilled in the art may easily practice the present invention. However, the present invention may be implemented in various forms and is not limited to the embodiments described herein. In the accompanying drawings, portions unrelated to the description will be omitted in order to obviously describe the present invention, and similar reference numerals will be used to describe similar portions throughout the present specification.

Throughout the specification, unless explicitly described to the contrary, the word “include” and variations such as “comprise” or “comprising” will be understood to imply the inclusion of stated elements but not the exclusion of any other elements.

Implementations described herein may be implemented in, for example, a method or process, an apparatus, a software program, a data stream, or a signal. Although discussed only in the context of a single form of implementation (e.g., discussed only as a method), implementations of the discussed features may also be implemented in other forms (for example, an apparatus or a program). The apparatus may be implemented in suitable hardware, software, firmware, and the like. A method may be implemented in an apparatus such as a processor, which is generally a computer, a microprocessor, an integrated circuit, a processing device including a programmable logic device, or the like.

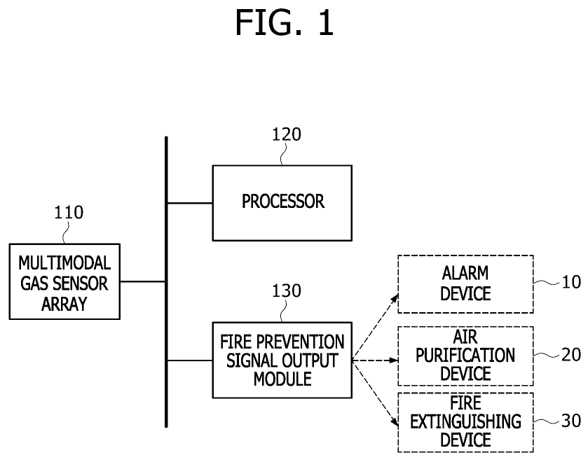

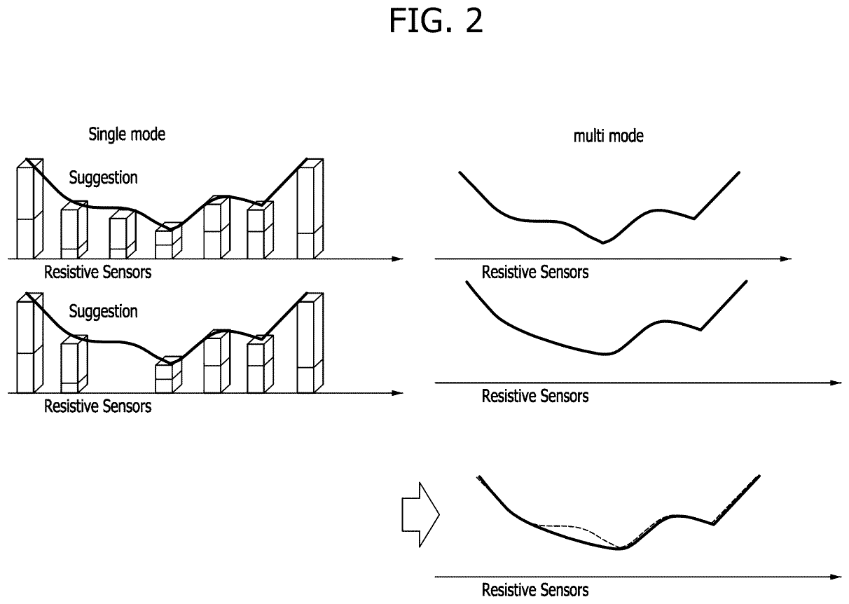

FIG. 1 is an exemplary view illustrating a schematic configuration of a fire detection apparatus using a multimodal gas sensor array according to one embodiment of the present invention. FIG. 2 is an exemplary view for describing the features of a gas detection pattern of a multimodal gas sensor array in FIG. 1.

As shown in FIG. 1, the fire detection apparatus using a multimodal gas sensor array according to the present embodiment includes a multimodal gas sensor array 110, a processor 120, and a fire prevention signal output module 130.

The multimodal gas sensor array 110 is not a single gas sensor (or a plurality of single gas sensors having the same sensitivity or detecting gas in the same manner) but refers to sensor arrays having different sensitivities, or gas sensor arrays operating in different modes (or different manners).

That is, the multimodal gas sensor array 110 detects a fire based on a gas detection pattern that may be detected by each gas sensor of the gas sensor array.

For reference, conventionally, as a sensor for fire detection, a single gas sensor has been used, or in addition to gas sensors such as an optical sensor, a smoke sensor, and a temperature sensor, a plurality of sensors capable of detecting various fire features have been used in combination.

However, these sensors (for example, a single gas sensor, an optical sensor, a smoke sensor, and a temperature sensor) operate to output a fire detection signal when a sensing value exceeds a predetermined threshold. Accordingly, when the sensing value is lower than the predetermined threshold, since a fire detection signal is not output, a fire alarm cannot be output.

However, in the present embodiment, a gas detection pattern is detected using the multimodal gas sensor array 110, and when a similar gas detection pattern is detected within an error range based on the detected gas detection pattern, a fire detection signal is output.

Accordingly, in the present embodiment, accurate fire detection can be performed irrespective of a failure or malfunction of individual gas sensors constituting the multimodal gas sensor array 110 or ambient noise affecting a specific gas sensor constituting the multimodal gas sensor array 110.

For reference, as in the related art, when a fire is to be detected using a single fire detection sensor (for example, a gas sensor, an optical sensor, a temperature sensor, a smoke sensor, or a sound field sensor) or a plurality of single fire detection sensors (for example, a plurality of single fire detection sensors that have the same sensitivity or detect fire features in the same manner), irrespective of the number of single fire detection sensors, each single fire detection sensor included in the plurality of single fire detection sensors cannot output a fire detection signal when a sensing value does not exceed a threshold.

As a result, the existing method has a problem that accurate fire detection cannot be determined because, in a plurality of single fire detection sensors (for example, a plurality of single fire detection sensors that have the same sensitivity or detect fire features in the same manner), the reliability and stability of each single fire detection sensor cannot be insured.

In addition, conventionally, even when using a combined sensor that detects different types of fire features at the same time (for example, a sensor in which a gas sensor, an optical sensor, a temperature sensor, and the like are integrally combined), there is a problem that accurate fire detection cannot be performed even when a failure or malfunction occurs in any one sensor that constitutes the combined sensor, and thus a sensing value of the combined sensor does not exceed a predetermined threshold.

However, unlike a related art, in the multimodal gas sensor array 110 according to the present embodiment, rather than by using a single gas sensor or a plurality of single gas sensors, by using gas sensor arrays having different sensitivities or operating in different modes (or different manners) as shown in FIG. 2, a fire is detected using a gas detection pattern that may be detected by each gas sensor.

In this case, even when a failure or malfunction occurs in an individual sensor or a plurality of sensors, the detection accuracy and selectivity for a specific gas generated by a fire can be improved. That is, even when there is a difference in sensing sensitivity between respective gas sensors included in the multimodal gas sensor array 110 according to the present embodiment, since gas detection patterns detected by the respective gas sensors have a similar form, a fire can be detected irrespective of a threshold.

The processor 120 may perform operations for executing various types of software, firmware, or program code. Through the multimodal gas sensor array 110, the processor 120 may measure a plurality of gases, in which various gases are mixed, generated when a fire occurs. Alternatively, one specific gas may be measured among a plurality of gases, in which various gases are mixed, generated when a fire occurs.

By using information (or data) detected using the multimodal gas sensor array 110, the processor 120 may pre-learn a gas detection pattern according to a position at which a fire has occurred (for example, a house, a factory, an energy storage system (ESS), or an electric vehicle), an object in which the fire has occurred (for example, wood, a synthetic compound, a chemical substance, paper, or a battery), and a fire range (for example, a fire range according to an initial stage, a middle stage, or a final stage of the fire).

Through an artificial intelligence model, the processor 120 may learn a detection pattern of gas (that is, gas in which various types of gases having various intensities are mixed) generated when a fire occurs. In this case, according to the sensitivity of an individual das sensor included in the multimodal gas sensor array 110, even when there is a difference in sensing sensitivity between respective gas sensors, gas detection patterns detected by the respective gas sensors have a similar form so that a fire can be detected irrespective of a threshold.

The processor 120 may perform various types of machine learning algorithms or deep learning algorithms such as a convolutional neural network (CNN) or a recurrent neural network (RNN).

The processor 120 may analyze a gas detection pattern to detect a fire even in an initial stage of fire occurrence.

The fire prevention signal output module 130 is a pre-designated device for fire response (or fire prevention) when the processor 120 determines (or detects) fire occurrence. The fire prevention signal output module 130 outputs a fire prevention signal to at least one of an alarm device 10, an air purification device 20, and a fire extinguishing device 30.

By using visual information and auditory information, the alarm device 10 may output a warning (or an alarm) including fire-related information (for example, a place/position at which a fire has occurred, an object in which the fire has occurred, a fire range/fire progression level, and the like).

The air purification device 20 may discharge gas or smoke, which is accumulated in an area (or position) in which a fire has occurred, to the outside, thereby allowing a view to be secured and allowing a fire to be prevented from spreading due to the accumulated gas or smoke.

The fire extinguishing device 30 may spray a fire extinguishing agent or a fire extinguishing powder corresponding to an object in which a fire has occurred (or a type of fire). In this case, a plurality of alarm devices 10, a plurality of air purification devices 20, and a plurality of fire extinguishing devices 30 may be provided.

However, in the present embodiment, a detailed description of a fire prevention method is omitted.

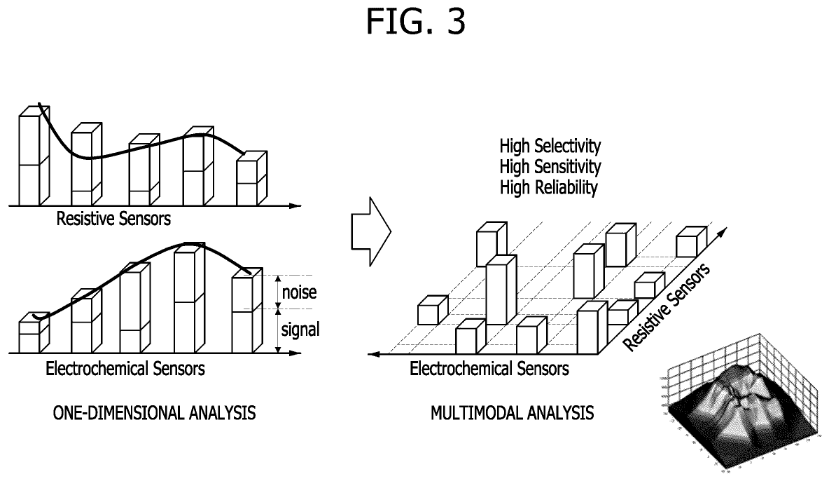

FIG. 3 is an exemplary view for describing an operating principle of the multimodal gas sensor array in FIG. 1.

Referring to FIG. 3, a pattern is used in a semiconductor metal oxide sensor array using a change in electrical resistance which is used generally and widely. Noise may be introduced according to a state of oxygen adsorption species on an intrinsic surface of a semiconductor metal oxide sensor and a surrounding environment (for example, temperature, humidity, and interfering gases).

When such a change in electrical resistance and other electrochemical sensor arrays are used, an electromotive force generated by an electrochemical reaction with a target gas is detected.

In this case, the influence of the surrounding environment such as temperature and interfering gases is small. However, sensitivity is low. In this case, noise in intrinsic odor detection occurs.

When sensor arrays of different types are provided together in this way, noise components resulting from external or surrounding environmental factors are removed, thereby forming more accurate multidimensional odor patterns.

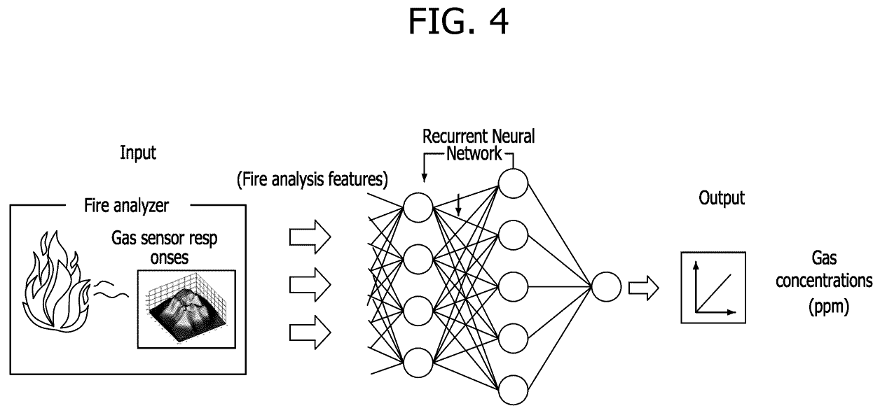

FIG. 4 is an exemplary view for describing a driving method in which artificial intelligence is introduced into the multimodal gas sensor array in FIG. 1.

Referring to FIG. 4, based on pre-performed learning (that is, learning performed through machine learning using an artificial intelligence mode), the processor 120 that receives information (or data) detected through the multimodal gas sensor array 110 may accurately quantify whether a fire has occurred or a concentration of odors (that is, gases) that are generated before the fire occurs.

That is, in the present embodiment, by using the multimodal gas sensor array 110, a fire can be quickly and accurately detected at an initial stage of fire occurrence based on odors.

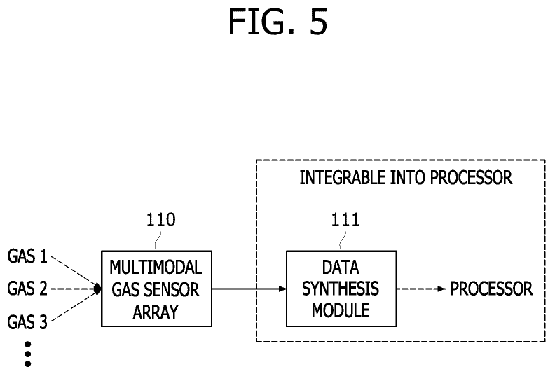

FIG. 5 is an exemplary view for describing an additional module for expanding a function of the multimodal gas sensor array in FIG. 1.

Referring to FIG. 5, each sensor included in the multimodal gas sensor array 110 may adjust a sensing condition thereof. Each sensor included in a sensor array may be implemented to include a plurality of gas sensors.

For example, each sensor array may be implemented to include sensor arrays including at least one of a metal oxide semiconductor (MOS) sensor, a photoionization detector sensor, a polymer sensor, an electrochemical Sensor (ECS), a microcantilever sensor, a surface plasmon resonance (SPR) sensor, and a catalytic combustion sensor.

The gas sensors may preferably be constituted by, for example, hydrogen sensors, carbon monoxide sensors, carbon dioxide sensors, and VOCs (Volatile Organic Compounds) sensors. The gas sensors may preferably be configured to detect within 10 minutes, the hydrogen sensor at a level of 1 to 100 ppm, the carbon monoxide sensor at a level of 1 to 100 ppm, the carbon dioxide sensor at a level of 1 to 1000 ppm, and the VOCs sensor at a level of 1 to 500 ppm, respectively. However, this configuration describes a preferred embodiment, and the gas sensor array may also be configured with some different arrangements as needed by those of ordinary skill in the art

In this case, the sensors included in the sensor array may have different modalities according to a method of implementing the sensors.

For example, a first sensor array may have a first modality based on sensors included in the first sensor array. For example, under the control of the processor 120, the characteristics of an input signal such as a voltage, a current, or a waveform of a control signal applied to each of the sensors included in the sensor array may vary.

Accordingly, sensing data associated with various modalities and various sensing conditions may be provided through each of the sensors included in the sensor array, and various types of gases may be detected under various conditions.

A data synthesis module 111 may synthesize multimodal sensing data detected by the multimodal gas sensor array 110. For example, the data synthesis module 111 may classify multimodal sensing data based on a target gas and synthesize the multimodal sensing data over time. However, the processor 120 may also integrate functions of the data synthesis module 111 and perform the integrated functions in software.

In the present embodiment, the processor 120 may perform fire detection by detecting odors (that is, gases) generated in an initial stage of fire occurrence by using a gas detection pattern detected through the multimodal gas sensor array 110.

For early warning in the event of a fire, to detect specific odors caused by the fire, a specific gas detection pattern is detected using the multimodal gas sensor array 110 implemented as gas sensor arrays that operate in different operating manners.

By analyzing specific odor detection signals (that is, specific gas detection patterns) based on artificial intelligence, it is possible to additionally detect whether a fire has occurred and a concentration of specific odor components.

A plurality of sensor arrays having characteristics such as a color change, an electrical resistance change, an electrochemical signal change, and an ion voltage change may be used in the multimodal gas sensor array 110 implemented as gas sensor arrays that operate in different operating manners.

However, the present invention is not limited to a specific operating mode, but rather, different modes may be integrated to remove noise, thereby more accurately detecting intrinsic signal patterns according to odors (that is, specific gas detection patterns corresponding to odors).

Thus, it is possible to obtain a stable discrimination signal which has accuracy and from which noise is removed.

Meanwhile, in the above-described embodiment, for convenience of description, the description focuses on the multimodal gas sensor array 110 for detecting gas generated when a fire occurs, but the present invention is not limited to gas detection. It is noted that, in other embodiments, the multimodal gas sensor array 110 may be implemented as a multimodal fire detection sensor array for detecting other types of characteristics generated when a fire occurs (for example, temperature detection, flame detection, sound field detection, or the like).

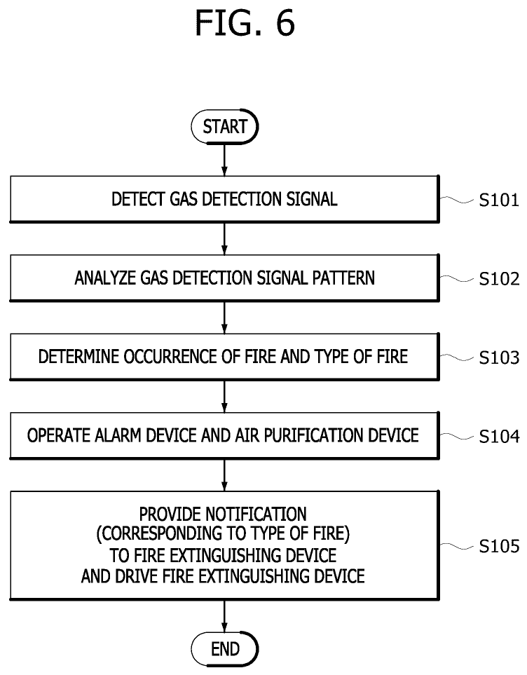

FIG. 6 is a flowchart for describing a fire detection method using a multimodal gas sensor array according to one embodiment of the present invention.

Referring to FIG. 6, the processor 120 recognizes that a gas detection signal detected through the multimodal gas sensor array 110 (S101).

The processor 120 analyzes a gas detection signal pattern detected through the multimodal gas sensor array 110 (S102) (see FIG. 2).

The processor 120 may analyze the gas detection signal pattern based on information (or data) detected using the multimodal gas sensor array 110 to determine whether a fire has occurred and a type of fire (for example, a type of fire including a position at which the fire has occurred, an object in which the fire has occurred, a fire range, and the like) (S103).

For example, by analyzing the gas detection signal pattern based on the information (or data) detected using the multimodal gas sensor array 110, the processor 120 may pre-perform learning for determining a position at which a fire has occurred (for example, a house, a factory, an ESS, or an electric vehicle), an object in which the fire has occurred (for example, wood, a synthetic compound, a chemical substance, paper, or a battery), and a fire range (for example, a fire range according to an initial stage, a middle stage, or a final stage of the fire).

When the processor 120 determines the occurrence of the fire and the type of fire, the processor 120 operates the alarm device 10 and the air purification device 20 through the fire prevention signal output module 130 (S104) and automatically provide a notification corresponding to the type of fire to the fire extinguishing device 30 and automatically drive the fire extinguishing device 30 (S105).

For example, the fire extinguishing device 30 may accurately extinguish a fire only when a fire extinguishing agent or a fire extinguishing powder corresponding to the type of fire is used. Therefore, fire occurrence may be simply notified through the alarm device 10, and information about which fire extinguishing device 30 is used to extinguish the fire may also be notified. In addition, when there is the fire extinguishing device 30 corresponding to the fire, the fire extinguishing device 30 may also be automatically driven.

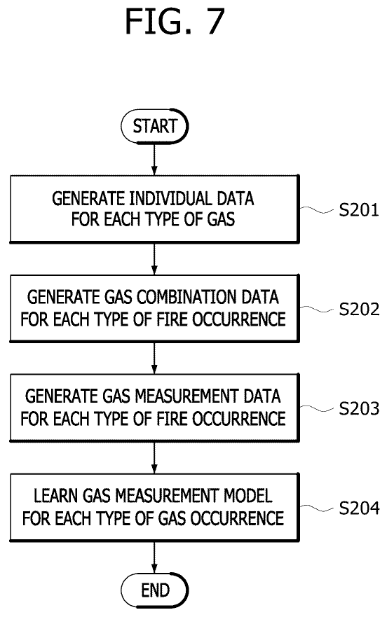

FIG. 7 is a flowchart for exemplarily describing a method of training the processor with a method of detecting a type of fire in FIG. 6.

Referring to FIG. 7, in order to train the processor 120 with the method of detecting a type of fire, a learning device (not shown) generates individual data for each type of gas (S201).

For example, the learning device (not shown) may generate individual data for each type of gas that may be generated according to a position at which the fire has occurred (for example, a house, a factory, an ESS, or an electric vehicle), an object in which the fire has occurred (for example, wood, a synthetic compound, a chemical substance, paper, or a battery), and a fire range (for example, a fire range according to an initial stage, a middle stage, or a final stage of the fire).

The learning device (not shown) generates gas combination data for each type of fire occurrence (S202).

For example, the learning device (not shown) may generate gas combination data that may be generated according to a type of fire (for example, a type of fire including a position at which the fire has occurred, an object in which the fire has occurred, a fire range, and the like).

The learning device (not shown) generates gas measurement data (for example, a concentration) for each type of fire occurrence (S203).

Accordingly, the learning device (not shown) may synthesize gas combination data and gas measurement data, which may be generated according to a type of fire (a type of fire including a position at which the fire has occurred, an object in which the fire has occurred, a fire range, and the like), over time, thereby generating a gas detection pattern that may be generated according to a type of fire.

The learning device (not shown) generates a gas measurement model for each type of fire occurrence based on a gas detection pattern that may be generated according to a type of fire, thereby allowing the processor 120 to learn the gas measurement model (S204).

In this way, according to the present embodiment, there is an effect in which a fire can be quickly and accurately detected even in an initial stage of fire occurrence by using a multimodal gas sensor array.

According to an aspect of the present invention, in the present invention, a fire can be quickly and accurately detected using a multimodal gas sensor array.

Although the present invention has been described with limited embodiments and drawings, the present invention is not limited to thereto, and instead, it would be appreciated by those skilled in the art that various modifications and changes may be made to these embodiments without departing from the principles and spirit of the present invention, the scope of which is defined by the claims and their equivalents.

What is claimed is:

1. A fire detection apparatus using a multimodal gas sensor array, comprising:
a multimodal gas sensor array configured to detect gas information when a fire occurs; and
a processor configured to analyze a gas detection pattern detected through the multimodal gas sensor array to determine occurrence of the fire and a type of the fire.

2. The fire detection apparatus of claim 1, wherein the multimodal gas sensor array is implemented as sensor arrays having different sensitivities or gas sensor arrays operating in different modes or in different manners.

3. The fire detection apparatus of claim 1, wherein the processor detects the fire based on the gas detection pattern that is detected by each gas sensor of a gas sensor array implemented in the multimodal gas sensor array.

4. The fire detection apparatus of claim 1, wherein the gas sensor array comprises a hydrogen sensor, a carbon monoxide sensor, a carbon dioxide sensor, and a VOCs (Volatile Organic Compounds) sensor.

5. The fire detection apparatus of claim 4, wherein the hydrogen sensor detects gas at a level of 1-100 ppm, the carbon monoxide sensor detects gas at a level of 1-100 ppm, the carbon dioxide sensor detects gas at a level of 1-1000 ppm, and the VOCs (Volatile Organic Compounds) sensor detects gas at a level of 1-500 ppm.

6. The fire detection apparatus of claim 1, wherein the processor determines the type of the fire using information detected by the multimodal gas sensor array, and
the type of the fire includes at least one of a position at which the fire has occurred, an object in which the fire has occurred, and a fire range.

7. The fire detection apparatus of claim 1, wherein, by using an artificial intelligence mode, the processor pre-learns a gas detection pattern of gas, which various types of gases having various intensities are mixed, generated when the fire occurs, and
even when there is a difference in sensing sensitivity between respective gas sensors included in the multimodal gas sensor array, gas detection patterns detected by the respective gas sensors have a similar form within an error range.

8. The fire detection apparatus of claim 1, further comprising a fire prevention signal output module configured to output a fire prevention signal to at least one fire prevention device for fire response, which is designated in advance, when the processor determines that the fire has occurred.

9. The fire detection apparatus of claim 8, wherein the fire protection device includes an alarm device, an air purification device, and a fire extinguishing device configured to spray a fire extinguishing liquid or fire extinguishing powder corresponding to an object in which the fire has occurred, or the type of the fire.

10. The fire detection apparatus of claim 1, wherein the processor analyzes a gas detection signal pattern detected through the multimodal gas sensor array based on pre-performed learning to determine the occurrence of the fire and the type of the fire, operates an alarm device and an air purification device through a fire protection signal output module when the occurrence of the fire and the type of the fire are determined, and also performs fire protection by automatically providing a notification corresponding to the type of the fire to a fire extinguishing device and automatically driving the fire extinguishing device.

11. The fire detection apparatus of claim 1, wherein the processor pre-learns a gas detection pattern that is generated according to the type of the fire provided by a learning device.

12. The fire detection apparatus of claim 11, wherein the learning device generates individual data for each type of gas, generates gas combination data for each type of fire occurrence, generates gas measurement data for each type of fire occurrence, and synthesizes gas combination data generated according to the type of the fire and gas measurement data over time to generate a gas detection pattern that is generated according to the type of the fire.

13. A fire detection method using a multimodal gas sensor array, comprising:
detecting, by a processor, gas information through a multimodal gas sensor array when a fire occurs; and
analyzing, by the processor, a gas detection pattern detected through the multimodal gas sensor array and determining occurrence of the fire and a type of the fire.

14. The fire detection method of claim 13, wherein, in the detecting of the gas information when the fire occurs, the multimodal gas sensor array is implemented as sensor arrays having different sensitivities or gas sensor arrays operating in different modes or in different manners.

15. The fire detection method of claim 13, wherein, in the determining of the occurrence of the fire and the type of the fire, the processor detects the fire based on the gas detection pattern that is detected by each gas sensor of a gas sensor array implemented in the multimodal gas sensor array.

16. The fire detection method of claim 13, wherein, in the determining of the occurrence of the fire and the type of the fire, the processor determines the type of the fire using information detected by the multimodal gas sensor array, and
the type of the fire includes at least one of a position at which the fire has occurred, an object in which the fire has occurred, and a fire range.

17. The fire detection method of claim 13, wherein, in the determining of the occurrence of the fire and the type of the fire, by using an artificial intelligence mode, the processor pre-learns a gas detection pattern of gas, which various types of gases having various intensities are mixed, generated when the fire occurs, and
even when there is a difference in sensing sensitivity between respective gas sensors included in the multimodal gas sensor array, gas detection patterns detected by the respective gas sensors have a similar form within an error range.

18. The fire detection method of claim 13, further comprising, after the determining of the occurrence of the fire and the type of the fire, outputting, by a fire prevention signal output module, a fire prevention signal to at least one fire prevention device for fire response, which is designated in advance, when the processor determines that the fire has occurred.

19. The fire detection method of claim 18, wherein, in the outputting of the fire prevention signal to the at least one fire prevention device, the fire protection device includes an alarm device, an air purification device, and a fire extinguishing device configured to spray a fire extinguishing liquid or fire extinguishing powder corresponding to an object in which the fire has occurred, or the type of the fire.

20. The fire detection method of claim 13, wherein, in the determining of the occurrence of the fire and the type of the fire, the processor analyzes a gas detection signal pattern detected through the multimodal gas sensor array based on pre-performed learning to determine the occurrence of the fire and the type of the fire, operates an alarm device and an air purification device through a fire protection signal output module when the occurrence of the fire and the type of the fire are determined, and also performs fire protection by automatically providing a notification corresponding to the type of the fire to a fire extinguishing device and automatically driving the fire extinguishing device.
